(19) United States
(12) Patent Application Publication
(10) Pub. No.: US 2015/0111973 A1
(43) Pub. Date: Apr. 23, 2015

(54) METHODS OF MODULATING STEROID HORMONE ACTIVITY

(71) Applicant: Novan, Inc., Durham, NC (US)

(72) Inventors: Susanne Bauman, Durham, NC (US); William Reed Kelce, Durham, NC (US)

(73) Assignee: Novan, Inc., Durham, NC (US)

(21) Appl. No.: 14/381,377

(22) PCT Filed: Feb. 28, 2013
(86) PCT No.: PCT/US2013/028215

§ 371 Date: Aug. 27, 2014

Related U.S. Application Data
(60) Provisional application No. 61/610,233, filed on Mar. 13, 2012.

(51) Int. Cl. A61K 9/00; A61K 31/275

(52) U.S. Cl. CPC A61K 9/0014; A61K 31/275
USPC 514/740

(57) ABSTRACT

The present invention relates to methods of treating steroid hormone related disorders by modulating steroid hormone activity.

### RELATED APPLICATION DATA

This application claims the benefit of U.S. Provisional Patent Application Ser. No. 61/610,233, filed Mar. 13, 2012, the disclosure of which is incorporated herein by reference in its entirety.

### FIELD OF THE INVENTION

The present invention relates to methods of treating steroid hormone related disorders by modulating steroid hormone activity in the skin of a subject.

### BACKGROUND OF THE INVENTION

Steroid hormones are involved in regulating various functions within the body. In particular, steroid hormones are involved in mechanisms within the skin and skin is a steroidogenic tissue meaning that the skin can produce and metabolize steroid hormones. Due to the involvement and presence of steroid hormones within the skin, steroid hormones may also affect skin related steroid hormone disorders. For example, the steroid hormone testosterone may be involved in acne. Acne vulgaris is the most common skin disease in the United States. It is estimated that 40 to 50 million Americans have acne, including 80% of people between the ages of 11 and 30. The annual direct costs associated with the treatment of acne exceeded $2.8 billion in 2007, with the majority of those costs attributable to prescription drugs. In addition, acne causes both physical and psychological effects, including permanent scarring, anxiety, depression, and poor self-esteem. Even in cases of mild acne, the social stigma associated with the disease frequently results in significant emotional distress and other psychological issues. Due to its social impact, frequency of recurrence or relapse, and necessary maintenance over a prolonged course of therapy, the American Academy of Dermatologists have recommend that acne vulgaris be re-classified and investigated as a chronic disease.

Acne vulgaris results from the interplay of four major pathogenic factors: 1) overproduction of sebum by the sebaceous gland; 2) abnormal keratinization in the follicle; 3) colonization of the hair follicles by the anaerobic, lipophilic bacterium Propionibacterium acnes, or P. acnes; and 4) release of inflammatory mediators into the skin.

Acne lesions begin when the combination of excess sebum and abnormal epithelial desquamation clog up a follicle, forming a microscopic lesion known as a microcomedo. The aneaerobic, lipid-rich environment of the microcomedo provides an ideal location for P. acnes proliferation. Each microcomedo may progress to form a non-inflammatory open or closed comedone (commonly referred to as a “blackhead” or “whitehead,” respectively), or an inflammatory lesion that may be further categorized as a papule, pustule, nodule, or cyst.

Multiple treatments that may span oral and topical antimicrobials, oral and topical retinoids, oral contraceptives and other prescription skin cleansers may be needed to treat the disease.

Antibiotics were the first successful acne treatment due to their antimicrobial and anti-inflammatory properties. Both topical and systemic antibiotics have been very successful, but the protracted treatment periods required have led to the development of resistance of P. acnes and in other non-targeted (and potentially pathogenic) commensal organisms. Combining antibiotics with topical retinoids targets three of the four major pathogenic factors associated with acne (all but sebum production). The oral retinoid isotretinoin (e.g., Accutane®) is the only drug known to affect all four pathogenic factors associated with acne. However, the severity of its potential side effects (known teratogen and linked to depression, psychosis and suicide) has limited its use and led to numerous lawsuits.

While the problems associated with isotretinoin are the most severe, all of the current acne medications have some adverse effects. The majority of topical treatments lead to dryness, irritation and peeling of the skin, and oral antibiotics may cause gastrointestinal tract irritation, photosensitivity of skin, headache, dizziness, anemia, bone and joint pain, nausea and/or depression. As such, new medications for the treatment of acne are desired, and particularly new treatments that target sebum production.

The present invention addresses previous shortcomings in the art by providing methods of treating steroid hormone related disorders by modulating steroid hormone activity.

### SUMMARY OF THE INVENTION

One aspect of the present invention comprises a method of treating a steroid hormone related disorder in the skin of a subject, the method comprising: administering a nitric oxide-releasing pharmaceutical composition to the skin of the subject to locally modulate steroid hormone activity in the skin of the subject, thereby treating said steroid hormone related disorder in the skin of the subject.

A second aspect of the present invention comprises a method of reducing steroid hormone induced sebum production in the skin of a subject, the method comprising: administering a nitric oxide-releasing pharmaceutical composition to the skin of the subject to locally modulate steroid hormone activity in the skin of the subject, thereby reducing sebum production in the skin of the subject.

A further aspect of the present invention comprises a method of reducing proliferation and/or differentiation of sebocytes and/or keratinocytes in the skin of a subject, the method comprising: administering a nitric oxide-releasing pharmaceutical composition to the skin of the subject to locally modulate steroid hormone activity in the skin of the subject, thereby reducing proliferation and/or differentiation of sebocytes and/or keratinocytes in the skin of the subject.

Another aspect of the present invention comprises a method of treating a disease of the pilosebaceous follicle in the skin of a subject comprising reducing androgen activity at a follicular androgen target in the skin of the subject by topically administering a nitric oxide-releasing pharmaceutical composition to the skin of the subject. In particular embodiments of the present invention, a nitric oxide-releasing pharmaceutical composition comprises a co-condensed silica macromolecule incorporating nitric oxide donor groups.

### BRIEF DESCRIPTION OF THE DRAWINGS

The following drawing is provided to illustrate various aspects of the present inventive concept and is not intended to limit the scope of the present invention unless specified herein.

FIG. 1 shows a graph of the results from a clinical study for non-inflammatory lesion reduction in males who received either a Vehicle composition or an Active composition according to an embodiment of the present invention.

FIG. 2 shows the in vitro nitric oxide release profile for a 2% Nitricil™ NVN1 Gel formulation over time.

FIG. 3 shows the in vitro nitric oxide release profiles for 2%, 6%, and 12% Nitricil™ NVN1 Gel formulations upon mixing with the hydrogel at pH 4 over time.

FIG. 4 shows the percent change in treated gland area over the course of treatment.

### DETAILED DESCRIPTION OF EMBODIMENTS OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of ±20%, ±10%, ±5%, ±1%, +0.5%, or even ±0.1% of the specified amount. A range provided herein for a measureable value may include any other range and/or individual value therein. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In the event of conflicting terminology, the present specification is controlling.

The embodiments described in one aspect of the present invention are not limited to the aspect described. The embodiments may also be applied to a different aspect of the invention as long as the embodiments do not prevent these aspects of the invention from operating for its intended purpose.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. Exemplary branched alkyl groups include, but are not limited to, isopropyl, isobutyl, tert-butyl. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₅ straight-chain alkyls. In other embodiments, “alkyl” refers, in particular, to C₁₋₅ branched-chain alkyls.

Alkyl groups may optionally be substituted (a “substituted alkyl”) with one or more alkyl group substituents, which may be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There may optionally be inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that may be a single aromatic ring, or multiple aromatic rings that are fused together, linked covalently, or linked to a common group, such as, but not limited to, a methylene or ethylene moiety. The common linking group also may be a carbonyl, as in benzophenone, or oxygen, as in diphenylether, or nitrogen, as in diphenylamine. The term “aryl” specifically encompasses heterocyclic aromatic compounds. The aromatic ring(s) may comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group may be optionally substituted (a “substituted aryl”) with one or more aryl group substituents, which may be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR¹R″, wherein R¹ and R″ may each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group may optionally be partially unsaturated. The cycloalkyl group also may be optionally substituted with an alkyl group substituent as defined herein, oxo, and/or alkylene. There may optionally be inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein may refer to, for example, methoxyl, ethoxyl, propoxyl, isopropoxyl, butoxyl, f-butoxyl, and pentoxyl. The term “oxyalkyl” may be used interchangeably with “alkoxyl”. In some embodiments, the alkoxyl has 1, 2, 3, 4, or 5 carbons.

“Aralkyl” refers to an aryl-alkyl group wherein aryl and alkyl are as previously described, and included substituted aryl and substituted alkyl. Exemplary aralkyl groups include benzyl, phenylethyl, and naphthylmethyl.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group may be straight, branched or cyclic. The alkylene group also may be optionally unsaturated and/or substituted with one or more “alkyl group substituents.” There may be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group may have about 2 to about 3 carbon atoms and may further have 6-20 carbons.

“Arylene” refers to a bivalent aryl group. An exemplary arylene is phenylene, which may have ring carbon atoms available for bonding in ortho, meta, or para positions with regard to each other, i.e., respectively. The arylene group may also be napthylene. The arylene group may be optionally substituted (a “substituted arylene”) with one or more “aryl group substituents” as defined herein, which may be the same or different.

“Aralkylene” refers to a bivalent group that contains both alkyl and aryl groups. For example, aralkylene groups may have two alkyl groups and an aryl group (i.e., -alkyl-aryl-alkyl-), one alkyl group and one aryl group (i.e., -alkyl-aryl-) or two aryl groups and one alkyl group (i.e., -aryl-alkyl-aryl-).

The term “amino” and “amine” refer to nitrogen-containing groups such as NR₃, NH₃, NHR₂, and NH₂R, wherein R may be alkyl, branched alkyl, cycloalkyl, aryl, alkylene, arylene, aralkylene. Thus, “amino” as used herein may refer to a primary amine, a secondary amine, or a tertiary amine. In some embodiments, one R of an amino group may be a cation stabilized diazeniumdiolate (i.e., NONO⁻X⁺).

The terms “cationic amine” and “quaternary amine” refer to an amino group having an additional (i.e., a fourth) group, for example a hydrogen or an alkyl group bonded to the nitrogen. Thus, cationic and quartemary amines carry a positive charge.

The term “alkylamine” refers to the -alkyl-NH₂ group.

The term “carbonyl” refers to the —(C═O)— group.

The term “carboxyl” refers to the —COOH group and the term “carboxylate” refers to an anion formed from a carboxyl group, i.e., —COO⁻.

The terms “halo”, “halide”, or “halogen” as used herein refer to fluoro, chloro, bromo, and iodo groups.

The term “hydroxyl” and “hydroxy” refer to the —OH group.

The term “hydroxyalkyl” refers to an alkyl group substituted with an —OH group.

The term “mercapto” or “thio” refers to the —SH group. The term “silyl” refers to groups comprising silicon atoms (Si).

As used herein the term “alkoxysilane” refers to a compound comprising one, two, three, or four alkoxy groups bonded to a silicon atom. For example, tetraalkoxysilane refers to Si(OR)₄, wherein R is alkyl. Each alkyl group may be the same or different. An “alkylsilane” refers to an alkoxysilane wherein one or more of the alkoxy groups has been replaced with an alkyl group. Thus, an alkylsilane comprises at least one alkyl-Si bond. The term “fluorinated silane” refers to an alkylsilane wherein one of the alkyl groups is substituted with one or more fluorine atoms. The term “cationic or anionic silane” refers to an alkylsilane wherein one of the alkyl groups is further substituted with an alkyl substituent that has a positive (i.e., cationic) or a negative (i.e. anionic) charge, or may become charged (i.e., is ionizable) in a particular environment (i.e., in vivo).

The term “silanol” refers to a Si—OH group.

The term “thiol” refers to a R—SH group.

One aspect of the present invention comprises, consists essentially of, or consists of a method of directly and/or indirectly locally modulating steroid hormone activity in the skin of a subject. Local modulation of a steroid hormone activity in the skin of a subject may be used to treat a steroid hormone related disorder in the skin of the subject. In some embodiments, a method of the present invention comprises administering a nitric oxide-releasing pharmaceutical composition to the skin of a subject to locally modulate steroid hormone activity in the skin of the subject, thereby treating a steroid hormone related disorder in the skin of the subject. In certain embodiments, the nitric oxide-releasing pharmaceutical composition is topically administered.

“Steroid hormone” as used herein refers to a natural and/or synthetic steroid hormone, steroid hormone precursor, steroid hormone metabolite, and/or derivative thereof that is structurally derived from cholesterol. Steroid hormones can be synthesized from cholesterol via pathways that involve cytochrome P450 (cP450) enzymes, which are heme-containing proteins. Exemplary steroid hormones, include, but are not limited to, androgens, estrogens, progestogens, mineralcorticoids, and glucocorticoids.

Exemplary androgens include, but are not limited to, testosterone, dehydroepiandrosterone, dehydroepiandrosterone sulphate, dihydrotestosterone, androstenedione, androstenediol, androstanedione, androstanediol, and any combination thereof.

Exemplary estrogens include, but are not limited to, estrone, estradiol, estriol, estetrol, equilin, equilenin, and any combination thereof.

Exemplary progestogens include, but are not limited to, progesterone, 17-hydroxy-progesterone, pregnenolone, dihydroprogesterone, allopregnanolone, 17-hydroxy-pregnenolone, 17-hydroxy-dihydroprogesterone, 17-hydroxy-allopregnanolone, and any combination thereof.

Exemplary mineralcorticoids include, but are not limited to, aldosterone, 11-deoxycorticosterone, fludrocortisones, 11-deoxy-cortisol, pregnenedione, and any combination thereof.

Exemplary glucocorticoids, include, but are not limited to, cortisol, corticosterone, 18-hydroxy-corticosterone, cortisone, and any combination thereof.

“Steroid hormone activity” as used herein refers to a steroid hormone's direct and/or indirect role and/or involvement in a chemical and/or biological mechanism within a subject, such as, but not limited to, a steroid hormone's involvement at a follicular steroid hormone target. “Follicular steroid hormone target” (e.g., follicular androgen target, follicular estrogen target, follicular glucocorticoid target, etc.) as used herein refers to sebocytes, keratinocytes, and/or the like on and/or in the skin of a subject that are affected by a steroid hormone and/or by the generation or production of a steroid hormone. In particular embodiments of the present invention, a follicular steroid hormone target is present in the skin of a subject.

For example, steroid hormone activity may refer to the production, metabolism, conversion, oxidation, and/or reduction of a steroid hormone. As one of ordinary skill in the art will recognize, the production, metabolism, conversion, oxidation, and/or reduction of a steroid hormone may involve other compounds, such as, but not limited to, enzymes. Thus, modulation of a steroid hormone's activity may comprise, for example, modulating the activity of an enzyme that produces and/or metabolizes a steroid hormone. Further, steroid hormone activity may comprise a steroid hormone's interactions (e.g., binding, signaling, etc.) with other components, such as but not limited to, steroid hormone receptors, proteins, etc. Thus, modulation of a steroid hormone's activity may comprise modulating the binding of a steroid hormone to a steroid hormone receptor and/or modulating a step in a signaling cascade. Exemplary steroid hormone receptors include, but are not limited to, peroxisome proliferation-activated receptors (PPAR), which may modulate sebocyte proliferation and/or differentiation, adrenodoxin reductase, steroidogenic factor 1, and/or any combination thereof. Adrenodoxin reductase and steroidogenic factor 1 may be found in human facial skin, human sebocytes, and/or SEB-1 sebocytes.

“Modulate,” “modulating,” “modulation,” and grammatical variations thereof as used herein refer to an increase or reduction in steroid hormone activity in the skin of a subject compared to the activity of the steroid hormone in the skin in the absence of a method of the present invention. As used herein, the terms “increase,” “increases,” “increased,” “increasing” and similar terms (e.g., activation, upregulation, and the like) indicate an elevation in activity of at least about 5%, 10%, 25%, 50%, 75%, 100%, 150%, 200%, 300%, 400%, 500% or more compared to the activity in the absence of a method of the present invention. As used herein, the terms “reduce,” “reduces,” “reduced,” “reduction” and similar terms (e.g., inhibition, downregulation, and the like) refer to a decrease in activity of at least about 5%, 10%, 25%, 35%, 50%, 75%, 80%, 85%, 90%, 95%, 97% or more compared to the activity in the absence of a method of the present invention.

A method of the present invention may directly modulate a steroid hormone activity by modulating an activity of the steroid hormone without the involvement of an intervening component and/or step. For example, direct modulation of a steroid hormone activity may involve modifying the steroid hormone in a manner that affects the activity of the steroid hormone and/or may involve affecting an activity of an enzyme that produces and/or metabolizes the steroid hormone. A method of the present invention may indirectly modulate a steroid hormone by modulating an activity of the steroid hormone that involves an intervening component and/or step. For example, indirect modulation of a steroid hormone activity may involve modifying a component that through two or more steps causes a change in the activity of the steroid hormone.

Exemplary steroid hormone activities that may be directly modulated by a method of the present invention, include, but are not limited to, modulation of nitrosylation of protein sulfhydryl groups, modulation of oxidation of iron-sulfur clusters, modulation of heme groups, and/or any combination thereof. “Nitrosylation” as used herein is differentiated from nitrosation. “Nitrosylation” as used herein refers to the formation of a nitroso group (i.e., R—N═O) at a metal center on a compound (e.g., an organic compound, a protein, a peptide, etc.) and thus results in a metal-N═O bond. For example, nitrosylation can occur on a heme protein, which comprises iron, to form an iron-N═O bond. In contrast, “nitrosation” as used herein refers to the formation of a nitroso group (i.e., R—N═O) on a compound (e.g., an organic compound, a protein, a peptide, etc.), but the nitroso group is not formed on a metal. Nitrosation may particularly occur on an amine and/or thiol functional group. For example, a protein comprising a thiol functional group (e.g., a cysteine) may be nitrosated to form a nitrosothiol functional group (i.e., R—S—N═O). In addition, whereas nitrosylation can occur directly from free nitric oxide gas, nitrosation cannot occur directly from free nitric oxide gas. Nitrosation may occur indirectly through N₂O₃, N₂O₄, and/or O═N—O—O—. Accordingly, as one of ordinary skill in the art will recognize, a compound, such as a protein, may be nitrosated and/or nitrosylated. For example, a heme protein could be both nitrosylated and nitrosated if the heme protein comprises both iron and, for example, an amino and/or thiol functional group. Nitrosylation and nitrosation each may alter a compound's structure and/or function. Nitrosation and/or nitrosylation may be modulated by a method of the present invention. In some embodiments, a steroid hormone and/or an enzyme that produces and/or metabolizes a steroid hormone may be nitrosated and/or nitrosylated.

Exemplary steroid hormone activities that may be indirectly modulated by a method of the present invention, include, but are not limited to, modulation of the activation of soluble guanylyl cyclase, modulation of a secondary messenger involved in a G protein signaling cascade, and/or any combination thereof. Modulation of soluble guanylyl cyclase (sGC) according to a method of the present invention may comprise nitric oxide that is released from the nitric oxide-releasing pharmaceutical composition binding to a heme group in sGC, thereby catalytically activating sGC.

“Local,” “locally,” and grammatical variations thereof, as used herein, refer to modulation of a steroid hormone activity and/or administration of a nitric oxide-releasing pharmaceutical composition of the present invention in and/or on the areas of the skin to which the composition is directly applied and to the areas in and/or on the skin to which a nitric oxide releasing active pharmaceutical ingredient present in the composition is configured to be delivered, such as, not limited to, areas in and/or on the skin that are outside of the administration site but to which the nitric oxide releasing active pharmaceutical ingredient may be delivered. Thus, local modulation of a steroid hormone activity refers to modulating a steroid hormone activity in and/or on the skin of a subject to which a nitric oxide-releasing active pharmaceutical ingredient in a composition of the present invention may be configured to be delivered. Local administration refers to administration of a nitric oxide-releasing active pharmaceutical ingredient in a composition of the present invention in and/or on the skin of a subject to which the nitric oxide-releasing active pharmaceutical ingredient may be configured to be delivered.

A nitric oxide-releasing active pharmaceutical ingredient in a composition of the present invention may be configured to deliver nitric oxide only to the skin of a subject. Thus, a nitric oxide-releasing active pharmaceutical ingredient in a composition of the present invention may not be systemically delivered in that nitric oxide does not cross the skin of a subject for systemic distribution of nitric oxide in the subject. In some embodiments of the present invention, local administration of a nitric oxide-releasing pharmaceutical composition of the present invention may have no adverse effects associated with systemic administration of a nitric oxide releasing active pharmaceutical ingredient present in the composition. In some embodiments of the present invention, local administration of a nitric oxide-releasing pharmaceutical composition of the present invention may have no systemic administration of a nitric oxide releasing active pharmaceutical ingredient present in the composition.

A method of the present invention may, in some embodiments, prevent and/or inhibit an enzyme, such as, but not limited to a cytochrome P450 enzyme, from directly and/or indirectly synthesizing or metabolizing a steroid hormone. For example, nitric oxide released from the nitric oxide-releasing pharmaceutical composition may bind to a cytochrome P450 enzyme and directly and/or indirectly inhibit production of a steroid hormone, such as, but not limited to, an androgen, an estrogen, a mineralocorticoid, and/or a glucocorticoid. In some embodiments of the present invention, nitric oxide released from a nitric oxide-releasing pharmaceutical composition may inhibit and/or alter the enzymatic structure and/or function of the side chain cleavage system, P450ssc, which is responsible for metabolizing cholesterol into pregnenolone. In other embodiments of the present invention, nitric oxide released from a nitric oxide-releasing pharmaceutical composition may modulate steroidogenesis in the skin of a subject in part through inhibition and/or alteration of protein structure and/or function of 17-α-hydroxylase/C_{17,20}-lyase (P450c17), the rate limiting enzymatic step for steroidogenesis.

In other embodiments of the present invention, modulation of steroid hormone activity according to a method of the present invention may directly and/or indirectly affect the sensitivity and/or responsiveness of a steroid hormone receptor to a steroid hormone. For example, in some embodiments of the present invention, the sensitivity of a steroid hormone receptor to a steroid hormone, such as, but not limited to, an androgen, may be reduced compared to the sensitivity of a steroid hormone receptor to the steroid hormone (e.g., androgen) in the absence of the methods of the present invention.

In certain embodiments of the present invention, a method of locally modulating steroid hormone activity in the skin of a subject is provided by topically administering a nitric oxide-releasing pharmaceutical composition to the skin of a subject. In particular embodiments of the present invention, methods are provided for locally modulating steroid hormone activity at follicular steroid hormone targets in the skin by topically administering a nitric oxide-releasing pharmaceutical composition to the skin of a subject.

In some embodiments of the present invention, the activity of an androgen, estrogen, and/or a glucocorticoid may be modulated at a follicular steroid hormone target. Both human sebocytes and keratinocytes in pilosebaceous follicles are targets for androgens and glucocorticoids. For example, sebaceous secretions may be stimulated by androgens and glucocorticoids. Also, sebocytes in the face multiply faster in the presence of testosterone and dihydrotestosterone. The sebocytes themselves may convert testosterone into dihydrotestosterone through the activity of 5α-reductase and, in particular, 5α-reductase Type I is found in the skin of the face and scalp. The sebocytes themselves may also produce dihydrotestosterone through the metabolism of androstanedione and androstandediol, a biosynthetic pathway known as the backdoor pathway of dihydrotestosterone synthesis. Another example includes the ability of estrogens to influence skin thickness, skin hydration, skin elasticity, and skin wrinkling by stimulating collagen synthesis, maturation, and turnover as well as keratinocyte proliferation.

Keratinocytes in the infra-infundibulum can metabolize androgens and, like the sebocytes of the face and scalp, 5α-reductase, Type I predominates in the keratinocytes in the infra-infundibulum. Other exemplary enzymes that may act to produce, metabolize, convert, oxidize, and/or reduce androgens include, but are not limited to, steroid sulphatases, 3β-hydroxy steroid dehydrogenase (e.g., type 1 and type 2), 3β-hydroxysteroid dehydrogenase/Δ⁵-Δ⁴ isomerase, 17β-hydroxysteroid dehydrogenase (e.g., types 1, 2, 3, 4, 6, 7, 8, 10, 11, 12, 13, and 14), 3α-hydroxysteroid dehydrogenase, aromatase, and any combination thereof. Androgen production and/or metabolism may have a substantial effect on keratinocyte proliferation and differentiation that could be associated with micro-comedo formation. Cortisol may also increase sebum production.

In some embodiments, a method of the present invention may comprise locally modulating steroid hormone activity in the skin of a subject to affect at least two of the major pathologies of a steroid hormone related disorder, such as, but not limited to, acne with the at least two pathologies being sebum production and hyper-keratinization. It has not previously been shown that nitric oxide can regulate steroid hormones in steroidogenic skin tissue. In some embodiments, a method of the present invention may comprise administering a NO-releasing pharmaceutical composition comprising an active ingredient consisting of at least one NO releasing API to the skin of a subject to locally modulate a steroid hormone activity. In some embodiments, the method may locally modulate a steroid hormone activity thereby decreasing sebum production in the skin of the subject and decreasing stimulation of the sebocyte by a steroid hormone, such as, but not limited to, an androgen, in the skin of the subject.

In particular embodiments, a method of the present invention may reduce androgen activity (e.g., testosterone activity or dihydrotestosterone activity) in the skin of a subject, such as, but not limited to, androgen activity at a follicular androgen target. In certain embodiments, a method of the present invention may comprise administering a nitric oxide-releasing pharmaceutical composition to the skin of a subject to locally reduce androgen activity in the skin of the subject. Reduction in androgen activity may be provided by a reduction in the synthesis of an androgen (e.g., by directly and/or indirectly inhibiting or reducing the activity of an enzyme that metabolizes or produces an androgen) and/or by inhibiting the activity of an androgen (e.g., by directly and/or indirectly inhibiting the binding of an androgen to an androgen receptor and/or blocking a step in a signaling cascade). While not being limited to any particular theory, it is postulated that nitric oxide may inhibit the synthesis of an androgen and/or another androgen activity in the skin and, thereby, reduce the production of sebum through a reduction in the rate of multiplication of sebocytes and/or through a reduction in the proliferation and/or differentiation of keratinocytes in the infra-infundibulum of pilosebaceous follicles. Accordingly, in some embodiments of the present invention, locally modulating androgen activity according to a method of the present invention may reduce sebum production and/or keratinocyte proliferation and/or differentiation in the skin of a subject. In some embodiments, a method of the present invention may reduce steroid hormone induced sebum production in the skin of a subject. “Steroid hormone induced sebum production,” as used herein refers to sebum production that is induced, activated, increased, and the like by a steroid hormone.

In other embodiments, a method of the present invention may comprise reducing glucocorticoid activity (e.g., cortisol activity) in the skin of a subject, such as, but not limited to, at a follicular glucocorticoid target. In some embodiments, a method of the present invention may comprise administering a nitric oxide-releasing pharmaceutical composition to the skin of a subject to locally reduce glucocorticoid activity in the skin of the subject. Reduction in glucocorticoid activity may be provided by a reduction in the synthesis of a glucocorticoid (e.g., by directly and/or indirectly inhibiting or reducing the activity of an enzyme that metabolizes a glucocorticoid) and/or by inhibiting the activity of a glucocorticoid (e.g., by directly and/or indirectly inhibiting the binding of a glucocorticoid to a glucocorticoid receptor and/or blocking a step in a signaling cascade).

In certain embodiments, a method of the present invention may comprise locally modulating a steroid hormone activity to thereby reduce sebum production in the skin of a subject. In some embodiments, a method of the present invention may comprise reducing sebum production by administering a nitric oxide-releasing pharmaceutical composition to the skin of a subject by locally modulating a steroid hormone activity. According to a method of the present invention, steroid hormone activity may be locally modulated in the skin of a subject to thereby treat a steroid hormone related disorder. Sebaceous glands are found in the skin and many sebaceous glands are connected to hair follicles. When stimulated by a steroid hormone, such as, but not limited to, an androgen, the sebaceous gland may increase in size and/or may secrete more sebum. As described in Giacomoni, Paolo U. et al, “Gender-linked differences in human skin” Journal of Dermatological Science, Vol. 55, Issue 5, September (2009), p. 144-149, the contents of which are incorporated herein by reference in its entirety, sebum production in men is generally higher than in women. In particular embodiments of the present invention, sebum production may be reduced by directly and/or indirectly inhibiting and/or preventing the synthesis of a steroid hormone, such as, but not limited to, an androgen and/or a glucocorticoid.

In other embodiments of the present invention, a method of reducing the proliferation and/or differentiation of sebocytes and/or keratinocytes may be provided comprising locally modulating steroid hormone activity by administering a nitric oxide-releasing pharmaceutical composition to the skin of a subject. In particular embodiments of the present invention, proliferation and/or differentiation of sebocytes and/or keratinocytes may be reduced by directly and/or indirectly inhibiting and/or preventing the synthesis of a steroid hormone, such as, but not limited to, an androgen and/or a glucocorticoid.

In some embodiments of the present invention, a method of locally modulating steroid hormone activity in the skin of a subject comprises, consists essentially of, or consists of administering at least one nitric oxide (NO) releasing active pharmaceutical ingredient (API) directly and/or indirectly to the skin of the subject. In certain embodiments of the present invention, the at least one NO releasing API is topically administered. Administering a nitric oxide releasing API “directly” refers to applying a NO releasing API directly onto the surface of the skin, without any barriers between the NO releasing API and the skin.

The term “nitric oxide releasing active pharmaceutical ingredient” refers to a compound, or other composition or device that provides nitric oxide to the skin, but is not gaseous nitric oxide. In some embodiments, the NO releasing API includes a nitric oxide-releasing compound, hereinafter referred to as a “NO-releasing compound.” An NO-releasing compound includes at least one NO donor, which is a functional group that may release nitric oxide under certain conditions. In certain embodiments, a nitric oxide-releasing pharmaceutical composition of the present invention only contains at least one nitric oxide releasing active pharmaceutical ingredient as the active pharmaceutical ingredient. Thus, in some embodiments of the present invention, while there may be two or more nitric oxide releasing active pharmaceutical ingredients in a composition of the present invention, the composition may not comprise an active pharmaceutical ingredient that is not a nitric oxide releasing active pharmaceutical ingredient.

Any suitable NO-releasing compound may be used. In some embodiments, the NO-releasing compound includes a small molecule compound that includes an NO donor group. “Small molecule compound” as used herein is defined as a compound having a molecular weight of less than 500 daltons, and includes organic and/or inorganic small molecule compounds. In some embodiments, the NO-releasing compound includes a macromolecule that includes an NO donor group, A “macromolecule” is defined herein as any compound that has a molecular weight of 500 daltons or greater. Any suitable macromolecule may be used, including crosslinked or non-crosslinked polymers, dendrimers, metallic compounds, organometallic compounds, inorganic-based compounds, and other macromolecular scaffolds. In some embodiments, the macromolecule has a nominal diameter ranging from about 0.1 nm to about 100 μm and may comprise the aggregation of two or more macromolecules, whereby the macromolecular structure is further modified with an NO donor group.

In particular embodiments of the present invention, an NO-releasing compound is lipophilic, such as, but not limited to, a lipophilic small molecule compound comprising an NO donor group and/or a lipophilic macromolecule comprising an NO donor group. A lipophilic NO-releasing compound may, in some embodiments of the present invention, allow the at least one NO donor to penetrate the skin of a subject. In certain embodiments of the present invention, a lipophilic NO-releasing compound may allow for the at least one NO donor to penetrate the skin of a subject to reach a sebaceous gland, an adrenal gland, and/or a gonad and/or may allow for release of NO at a sebaceous gland, an adrenal gland, and/or a gonad.

In some embodiments of the invention, the NO donor of the NO-releasing compound releases nitric oxide upon exposure to an external condition, such as light, heat, water, acid, base, and the like. For example, in some embodiments, the NO-releasing compound includes a diazeniumdiolate functional group as an NO donor. The diazeniumdiolate functional group may produce nitric oxide under certain conditions, such as upon exposure to water. As another example, in some embodiments, the NO-releasing compound includes a nitrosothiol functional group as the NO donor. The NO donor may produce nitric oxide under certain conditions, such as upon exposure to light. Examples of other NO donor groups include nitrosamine, hydroxyl nitrosamine, hydroxyl amine and hydroxyurea. Any suitable combination of NO donors and/or NO-releasing compounds may also be used in the methods described herein. Additionally, the NO donor may be incorporated into or onto the small molecule or macromolecule through covalent and/or non-covalent interactions.

In some embodiments of the invention, the NO donor of the NO-releasing compound releases nitric oxide upon exposure to an external condition, such as light, heat, water, acid, base, and the like. For example, in some embodiments, the NO-releasing compound includes a diazeniumdiolate functional group as an NO donor. The diazeniumdiolate functional group may produce nitric oxide under certain conditions, such as upon exposure to water. As another example, in some embodiments, the NO-releasing compound includes a nitrosothiol functional group as the NO donor. The NO donor may produce nitric oxide under certain conditions, such as upon exposure to light. Examples of other NO donor groups include nitrosamine, hydroxyl nitrosamine, hydroxyl amine and hydroxyurea. Any suitable combination of NO donors and/or NO-releasing compounds may also be used in the methods described herein. Additionally, the NO donor may be incorporated into or onto macromolecule through covalent and/or non-covalent interactions.

In some embodiments of the invention, the NO-releasing macromolecules may be in the form of NO-releasing particles, such as those described in U.S. Pat. No. 8,282,967, the disclosure of which is incorporated by reference herein in its entirety. Such particles may be prepared by methods described therein.

Any suitable NO-releasing compound that provides macromolecular delivery of nitric oxide may be used. The NO-releasing compound may release nitric oxide by any suitable mechanism, including via reaction with water and/or thermal degradation. Examples of NO-releasing functional groups that may be included in the NO-releasing compound include, but are not limited to, diazeniumdiolate, nitrosamine, hydroxyl nitrosamine, nitrosothiol, hydroxyl amine, hydroxyurea, and metal nitrosyl complexes. Other NO-releasing functional groups that are capable of releasing nitric oxide in a therapeutic manner, such as acidified nitrite, may also be utilized.

The NO-releasing compound may be a small molecule compound, an oligomer and/or a polymer and may be in any suitable physical form, such as, but not limited to, a particle, coating, film, liquid, solution and the like. In some embodiments, the nitric oxide-releasing compound comprises diazeniumdiolate-functionalized polysiloxane macromolecules as described above. Other non-limiting examples of NO-releasing compounds include NO-releasing zeolites as described in United States Patent Publication Nos. 2006/0269620 or 2010/0331968; NO-releasing metal organic frameworks (MOFs) as described in United States Patent Application Publication Nos. 2010/0239512 or 2011/0052650; NO-releasing multi-donor compounds as described in International Application No. PCT/US2012/52350 entitled “Tunable Nitric Oxide-Releasing Macromolecules Having Multiple Nitric Oxide Donor Structures”; NO-releasing dendrimers or metal structures as described in U.S. Publication No. 2009/0214618; nitric oxide releasing coatings as described in U.S. Publication No. 2011/0086234; and compounds as described in U.S. Publication No. 2010/0098733. The disclosures of each of the references in this paragraph are incorporated herein by reference in their entirety. Additionally, NO-releasing macromolecules may be fabricated as described in International Publication No. WO 2012/100174 entitled “Temperature Controlled Sol-Gel Co-Condensation” filed Jan. 20, 2012, the disclosure of which is incorporated herein by reference in its entirety.

As an example, in some embodiments of the invention, the NO-releasing particles include NO-loaded precipitated silica. The NO-loaded precipitated silica may be formed from nitric oxide donor modified silane monomers into a co-condensed siloxane network. In one embodiments of the invention, the nitric oxide donor is an N-diazeniumdiolate.

In some embodiments, the nitric oxide donor may be formed from an aminoalkoxysilane by a pre-charging method, and the co-condensed siloxane network may be synthesized from the condensation of a silane mixture that includes an alkoxysilane and the aminoalkoxysilane to form a nitric oxide donor modified co-condensed siloxane network. As used herein, the “pre-charging method” means that aminoalkoxysilane is “pretreated” or “precharged” with nitric oxide prior to the co-condensation with alkoxysilane. In some embodiments, the precharging nitric oxide may be accomplished by chemical methods. In another embodiment, the “pre-charging” method may be used to create co-condensed siloxane networks and materials more densely functionalized with NO-donors.

The co-condensed siloxane network may be silica particles with a uniform size, a collection of silica particles with a variety of size, amorphous silica, a fumed silica, a nanocrystalline silica, ceramic silica, colloidal silica, a silica coating, a silica film, organically modified silica, mesoporous silica, silica gel, bioactive glass, or any suitable form or state of silica.

In some embodiments, the alkoxysilane is a tetraalkoxysilane having the formula Si(OR)₄, wherein R is an alkyl group. The R groups may be the same or different. In some embodiments the tetraalkoxysilane is selected as tetramethyl orthosilicate (TMOS) or tetraethyl orthosilicate (TEOS). In some embodiments, the aminoalkoxysilane has the formula: R″—(NH—R′)ₙ—Si(OR)₃, wherein R is alkyl, R′ is alkylene, branched alkylene, or aralkylene, n is 1 or 2, and R″ is selected from the group consisting of alkyl, cycloalkyl, aryl, and alkylamine.

In some embodiments, the aminoalkoxysilane may be selected from N-(6-aminohexyl)aminopropyltrimethoxysilane (AHAP3); N-(2-aminoethyl)-3-aminopropyltrimethoxysilane (AEAP3); (3-trimethoxysilylpropyl)di-ethylenetriamine (DET3); (aminoethylaminomethyl)phenethyltrimethoxysilane (AEMP3); [3-(methylamino)propyl]trimethoxysilane (MAP3); N-butylamino-propyltrimethoxysilane(n-BAP3); t-butylamino-propyltrimethoxysilane (t-BAP3); N-ethylaminoisobutyltrimethoxysilane (EAiB3); N-phenylamino-propyltrimethoxysilane (PAP3); and N-cyclohexylaminopropyltrimethoxysilane (cHAP3).

In some embodiments, the aminoalkoxysilane has the formula: NH [R′—Si(OR)₃]₂, wherein R is alkyl and R′ is alkylene. In some embodiments, the aminoalkoxysilane may be selected from bis(3-triethoxysilylpropyl)amine, bis[3-(trimethoxysilyl)propyl]amine and bis-[(3-trimethoxysilyl)propyl]ethylenediamine.

In some embodiments, as described herein above, the aminoalkoxysilane is precharged for NO-release and the amino group is substituted by a diazeniumdiolate. Therefore, in some embodiments, the aminoalkoxysilane has the formula: R″—N(NONO⁻X⁺)—R′—Si(OR)₃, wherein R is alkyl, R′ is alkylene or aralkylene, R″ is alkyl or alkylamine, and X⁺ is a cation selected from the group consisting of Na⁺, K⁺ and Li⁺.

The composition of the siloxane network, (e.g., amount or the chemical composition of the aminoalkoxysilane) and the nitric oxide charging conditions (e.g., the solvent and base) may be varied to optimize the amount and duration of nitric oxide release. Thus, in some embodiments, the composition of the silica particles may be modified to regulate the half-life of NO release from silica particles.

In another embodiment, the amino group of aminoalkoxysilane is substituted with a diazeniumdiolate, and the aminoalkoxysilane having a formula of R″—N(NONO⁻X⁺)—R′—Si(OR)₃, wherein: R is alkyl, R′ is alkylene or aralkylene, R″ is alkyl or alkylamine, and X⁺ is a cation selected from the group consisting of Na⁺ and K⁺.

In some embodiments of the present invention, the particle size of a NO-releasing particle is in a range of about 20 nm to about 10 μm. The particle size may be tailored to minimize and/or prevent toxicity and penetration through the epidermis (or compromised dermis) and/or into the blood vessels. In particular embodiments of the present invention, the particle size is distributed around a mean particle size of less than about 10 μm to allow the particle to enter a follicle. In further embodiments of the present invention, the particle size is distributed around a mean particle size of less than about 8 μm. In other embodiments of the present invention, the particle size is distributed around a mean particle size of greater than about 10 μm to prevent the particle from entering the follicle.

In still further embodiments of the present invention, a mixture of particles with mean particle sizes distributed around two or more mean particle sizes may be provided. For example, a mixture of particles having a particle size distributed around a mean particle size of less than about 10 μm to allow the particle to enter a follicle may be mixed with particles having a particle size distributed around a mean particle size of greater than about 10 μm to prevent the particle from entering the follicle. The particles may have the same nitric oxide release profiles or different nitric oxide release profiles.

In certain embodiments of the present invention, the concentration of nitric oxide delivered and/or the rate of nitric oxide release may be controlled and/or varied, thereby the modulation of steroid hormone activity may be controlled and/or varied based on the nitric oxide delivered and/or the rate of nitric oxide release.

In some embodiments, at least one NO-releasing compound is applied to the skin in a pharmaceutically acceptable composition. As such, at least one NO releasing API is present in the pharmaceutically acceptable compositions. A pharmaceutically acceptable composition, as defined herein, refers to a composition that is suitable for application to a subject, such as a human, without undue side effects such as toxicity or irritation to the skin. Undue side effects are those that render the composition unsuitable for application to a subject because the harm from the side effects outweighs the benefits of the composition. In some embodiments, pharmaceutically acceptable compositions include at least one NO-releasing compound; optionally, at least one additional therapeutic agent; and at least one pharmaceutically acceptable excipient. In particular embodiments of the present invention, a pharmaceutical composition comprises a lipophilic nitric oxide-releasing compound. In certain embodiments of the present invention, a pharmaceutical composition comprises a nitric oxide-releasing compound and one or more lipophilic excipients, optionally providing a lipophilic pharmaceutical composition.

A NO-releasing compound may be present in a pharmaceutically acceptable composition according to embodiments of the invention at any suitable concentration. In some embodiments, a NO-releasing compound is present in a pharmaceutically acceptable composition of the present invention in an amount effective to modulate a steroid hormone activity in a subject. In some embodiments, a NO-releasing compound is present in a pharmaceutically acceptable composition of the present invention at a concentration sufficient to decrease, eliminate or prevent acne and/or decrease sebum production. In some embodiments, the concentration of a NO-releasing compound in a composition of the present invention is about 0.1% to about 30% w/w in the composition or any range and/or individual value therein, such as, but not limited to about 0.5% to about 20% or about 1% to about 10% w/w in the composition.

In some embodiments, a composition of the present invention comprises nitric oxide concentrations in an amount of about 0.001 μmol NO/mg of the composition to about 1.0 μmol NO/mg of the composition or any range and/or individual value therein, and in particular embodiments in an amount of about 0.006 μmol NO/mg of the composition to about 0.1 μmol NO/mg of the composition. An NO-releasing compound may be present in a composition of the present invention in an amount sufficient to deliver about 0.001 μmol NO/mg of the composition to about 1.0 μmol NO/mg of the composition or any range and/or individual value therein.

The pharmaceutically acceptable compositions may be present in any physical form, such as ointments, creams, milks, pomades, powders, impregnated pads, solutions, gels, sprays, lotions or suspensions. They may also be in the form of suspensions of microspheres or nanospheres or of lipid or polymeric vesicles, or of polymeric patches and hydrogels for controlled release. These compositions for topical application may be in anhydrous form, in aqueous form or in the form of an emulsion (e.g., oil in water or water in oil emulsions).

The term excipient refers to “inert” constituents of pharmaceutically acceptable compositions. The term “inert” indicates that such constituents are not therapeutic agents such as NO-releasing compounds or other antimicrobial compounds, anti-inflammatory agents, pain-relievers, immunosuppressants and vasodilators. However, as one of ordinary skill in the art will understand, the excipients may provide beneficial or therapeutic action to the skin (e.g., moisturize.) that may directly affect the acne. The excipients may also indirectly affect the treatment of acne by affecting the activity of the NO-releasing compounds or other therapeutic agents (i.e., active pharmaceutical ingredients) within the compositions.

Excipients for use in topical formulations are well-known in the art and examples may be found in the Handbook of Pharmaceutical Excipients (Rowe, R. C. et al., APhA Publications; 5^{th} ed., 2005). Exemplary excipients may include talc, calcium carbonate, calcium phosphate, magnesium stearate, waxes, various sugars and types of starch, polymers, gels, emollients, thickening agents, rheology modifiers, humectants, glycerol, organic basic compounds, cellulose derivatives, gelatin, vegetable oils, polyethylene glycols and solvents. Examples of rheology modifiers include Carbopol, C₂₆₋₂₈ alkyl dimethicone, C₂₆₋₂₈ alkyl methicone, polyphenylsisquioxane, trimethylsiloxysilicate, crosspolymers of cyclopentasiloxane and dimethicone/vinyltrimethylsiloxysilicate, and mixtures thereof. Examples of emollients include glycerine, pentylene glycol, sodium pyrrolidone carboxylic acid, lanolin, saccharide isomerate, stearoxy dimethicone, stearyl dimethicone, and mixtures thereof. Emollients may be useful to prevent stratum corneum dehydration occurring due to the use of anhydrous solvents in the formulation. Examples of organic bases include methanolamines, triethanolamines, Trisamino, AMP-95, AmP-Ultra PC 2000, triisopropanolamine, diisopropanolamine, Neutrol TE, Ethomeen, and mixtures thereof. The organic base may render the pH of the medicament basic or neutral, and may directly affect the release of NO from the NO-releasing compounds that include diazeniumdiolate NO donor groups by slowing donor decomposition with increasing alkalinity.

Other exemplary excipients include water-soluble porogens. A water-soluble porogen is an additive that may facilitate water uptake and diffusion into the pharmaceutically acceptable composition. Any suitable porogen may be used, but in some embodiments, the porogen may include sodium chloride, sucrose, glucose, lactose, sorbitol, xylitol, polyethylene glycol, polyvinylpyrrollidone, polyvinyl alcohol or mixtures thereof. Electrolytes, such as NaCl, may also be added as excipients.

Polymers may also act as excipients. Exemplary polymers include hydrophilic polyurethanes, hydrophilic polyacrylates, co-polymers of carboxymethylcellulose and acrylic acid, N-vinylpyrrolidone, poly(hydroxy acids), polyanhydrides, polyorthoesters, polyamides, polycarbonates, polyalkylenes (e.g., polyethylene and polypropylene), polyalkylene glycols (e.g., poly(ethylene glycol)), polyalkylene oxides (e.g., polyethylene oxide), polyalkylene terephthalates (e.g., polyethylene terephthalate), polyvinyl alcohols, polyvinyl ethers, polylvinyl esters, polyvinyl halides (e.g., poly(vinyl chloride)), polyvinylpyrrolidone, polysiloxanes, poly(vinyl acetates), polystyrenes, polyurethane copolymers, cellulose, derivatized celluloses, alginates, poly(acrylic acid), poly(acrylic acid) derivatives, acrylic acid copolymers, methacrylic acid, methacrylic acid derivatives, methacrylic acid copolymers, poly(butyric acid), poly(valeric acid), poly(lactide-co-caprolactone), copolymers thereof and blends thereof.

In some embodiments of the invention, the polymers may be superabsorbent polymers (SAPs). A polymer is considered superabsorbent, as defined per IUPAC, as a polymer that may absorb and retain extremely large amounts of water relative to its own mass. SAPs may absorb water up to 500 times their own weight and may swell up to 1000-times their original volume. Particular SAPs of interest include sodium polyacrylate, the polyurethane Tecophilic TG-2000, and polymers prepared by the use of polyacrylamide copolymer, ethylene maleic anhydride copolymer, cross-linked carboxy-methyl-cellulose, polyvinyl alcohol copolymers, polyvinylpyrrolindone and cross-linked polyethylene oxide. In some embodiments, the SAP may absorb water from the skin, thereby causing NO to release from the NO-releasing compounds.

In some embodiments of the invention, polymers that are relatively hydrophobic may be used. Any suitable hydrophobic polymer may be used. However, exemplary polymers that are relatively hydrophobic include aromatic polyurethanes, silicone rubber, polysiloxanes, polycaprolactone, polycarbonate, polyvinylchloride, polyethylene, poly-L-lactide, poly-DL-glycolide, polyetheretherketone (PEEK), polyamide, polyimide and polyvinyl acetate. In addition, a hydrophobic gel-base and/or rheology modifier may be used.

In some embodiments of the invention, notably in gels, the polymers may act as thickening agents in the medicaments. Specifically, the polymeric portion of the gel may act as a visco-elastic substance and may retain the gel at the site of application, along with the NO-releasing compounds dispersed therein.

In some other embodiments, notably in gels and ointments, a medicament that includes a polymer may have spreadability such that it forms a thin film when applied on the skin surface. This film may enable the application of the contained NO-releasing compounds over a wide area, and may serve to maintain the NO-releasing compounds on the affected area of the skin.

Other excipients may include various ionic or non-ionic compounds to maintain stability of the formulation, thereby protecting from the de-emulsification, settling, agglomeration or degradation of the formulation constituents that may reduce its therapeutic or aesthetic value.

Examples of ionic compounds may include salts such as sodium chloride, potassium chloride; cationic, anionic or zwitterionic surfactants such as sodium dodecyl sulfate (SDS), perfluorooctanoate (PFOA), perfluorooctanesulfonate (PFOS), ammonium lauryl sulfate (ALS), sodium lauryl ether sulfate (SLES), alkyl benzene sulfonate, cetyl trimethylammonium bromide (CTAB), cetylpyridinium chloride (CPC), polyethoxylated tallow amine (POEA), benzalkonium chloride (BAC), benzethonium chloride, dodecyl betaine, cocamidopropyl betaine and cocoamphoglycinate.

Examples of non-ionic compounds that may act as excipients include non-ionic surfactants such as Pluronic, Tween, AMP, and Brij family of surfactants; and surfactants derived from biological sources, e.g., natural or semi-synthetic surfactants, such as oleic acid, sorbitan trioleate, sorbitan monooleate, lecithin, cocamide MEA, cocamide DEA and cocamidopropyl betaine. Surfactants (both ionic and non-ionic) may reduce the interfacial surface energy and may facilitate spreading of the ointment or liquid over a wider area.

In some embodiments of the invention, solvent excipients may be used as a carrier vehicle for the NO-releasing compounds and other excipients. The polymer chains may interact with the solvent and undergo swelling to form a network that may impart visco-elastic properties to the medicament. In some embodiments of the medicament, the solvent may evaporate upon application, leaving a residual film of the polymer along with the entrapped NO-releasing compounds.

Examples of solvent excipients include dimethyl isosorbide, propylene glycol, glycerol, isopropanol, ethanol, ethylene glycol, polyethylene glycol, ethoxydiglycol or mixtures thereof. Exemplary solvent excipients that may be useful in hydrophobic formulations may include isododecane, isodecyl neopentanoate, butylene glycol, pentylene glycol, hexylene glycol, methoxypolyethyleneglycol, cyclopentasiloxane, cyclotetrasiloxane, dimethicone, caprylyl methicone or mixtures thereof.

In addition to the NO-releasing molecules, excipients, and other therapeutic agents, the pharmaceutically acceptable compositions may also include other compounds that improve the organoleptic properties of the composition. Examples of such compounds include perfumes, dyes and colorants; chelating agents including but not limited to EDTA, EGTA, CP94, citric acid; preservatives including but not limited to quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

A nitric oxide-releasing pharmaceutical composition described herein may comprise any suitable pharmaceutical composition comprising at least one NO-releasing compound. Exemplary nitric oxide-releasing pharmaceutical compositions include, but are not limited to, those described in International Publication No. WO 2011/022652, U.S. Provisional Application Ser. No. 61/552,395 entitled “Nitric Oxide Releasing Bath Compositions and Methods of Using the Same” filed Oct. 27, 2011, and U.S. Provisional Application Ser. No. 61/504,628 entitled “Topical Compositions and Methods of Using the Same to Treat Acne” filed Jul. 5, 2011 and U.S. Provisional Patent Application Ser. No. 61/610,137 (Attorney Docket No. 9729-25PR2) entitled “Topical Compositions” filed Mar. 13, 2012, the disclosures of each of which are incorporated herein by reference in their entirety. In addition, a nitric oxide-releasing pharmaceutical composition described herein may be prepared as described in U.S. Provisional Patent Application Ser. No. 61/504,626 and U.S. Provisional Patent Application Ser. No. 61/610,179 (Attorney Docket No. 9729-26PR2) both entitled “Methods of Manufacturing Topical Compositions and Apparatus For Same” filed Jul. 5, 2011 and Mar. 13, 2012, respectively, the disclosures of each of which is incorporated herein by reference in their entirety.

As discussed above, provided according to embodiments of the present invention are methods of locally modulating steroid hormone activity in the skin of a subject comprising administering a nitric oxide-releasing pharmaceutical composition to the skin of the subject, thereby treating a steroid hormone related disorder. In some embodiments, the method comprises topically administering the NO-releasing pharmaceutical composition to the skin of the subject. A method of the present invention may locally modulate steroid hormone synthesis in the skin of a subject. In particular embodiments of the present invention, methods are provided for modulating steroid hormone activity (e.g., androgen, estrogen, and/or glucocorticoid activity) in a pilosebaceous follicle in the skin of a subject by topically administering a nitric oxide-releasing pharmaceutical composition to the skin of a subject. In certain embodiments, a method of the present invention may reduce sebum production and/or the proliferation and/or differentiation of sebocytes and/or keratinocytes in the skin of a subject.

“Topical administration” and grammatical variants as used herein refer to a mode of applying a nitric oxide-releasing pharmaceutical composition of the present invention onto the skin of a subject such that an active ingredient in the composition, such as a nitric oxide releasing active pharmaceutical ingredient, may be delivered onto and/or into the skin of the subject. Topical administration is distinguished from transdermal administration in which the active ingredient is delivered across the skin providing for systemic delivery and/or distribution of the active ingredient. Thus, topical administration includes delivery of an active ingredient to one or more layers of the skin, such as, but not limited to, the epidermis and/or dermis, but does not include delivery of the active ingredient across through the skin for systemic delivery and/or distribution.

The present invention finds use in both veterinary and medical applications. Subjects suitable to be treated with a method embodiment of the invention include, but are not limited to, avian and mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult. In certain embodiments of the present invention, the subject is a human adolescent and/or adult male. In other embodiments of the present invention, the subject is a human adolescent and/or adult female.

Illustrative avians according to the present invention include chickens, ducks, turkeys, geese, quail, pheasant, ratites (e.g., ostrich) and domesticated birds (e.g., parrots and canaries), and birds in ovo.

The methods of the present invention may also be carried out on animal subjects, particularly mammalian subjects such as mice, rats, dogs, cats, livestock and horses for veterinary purposes, and/or for drug screening and drug development purposes.

In particular embodiments of the present invention, the subject is “in need of” a method of the present invention, e.g., the subject has been diagnosed with a disease, disorder, and/or condition associated with a steroid hormone, the subject is at risk for a disease, disorder, and/or condition associated with a steroid hormone, or it is believed that the subject has a disease, disorder, and/or condition associated with a steroid hormone. In certain embodiments, the disease, disorder, and/or condition associated with a steroid hormone is a skin disease, disorder, and/or condition associated with a steroid hormone. In some embodiments of the present invention, the subject has been diagnosed with a skin disease, disorder, and/or condition associated with a steroid hormone, such as, but not limited to an androgen (e.g. testosterone) and/or a glucocorticoid (e.g., cortisol).

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom is achieved and/or there is a delay in the progression of the skin disease, disorder, and/or condition. In particular embodiments of the present invention, the severity of the disease, disorder, and/or condition is reduced in a subject compared to the severity of the disease, disorder, and/or condition in the absence of the methods of the present invention.

In certain embodiments of the present invention, a therapeutically effective amount of nitric oxide is delivered to a subject. As used herein, the term “therapeutically effective amount” refers to an amount of a nitric oxide that elicits a therapeutically useful response in a subject. Those skilled in the art will appreciate that the therapeutic effects need not be complete or curative, as long as some benefit is provided to the subject.

Local modulation of a steroid hormone activity may be a treatment for a steroid hormone related disease, disorder, and/or condition such as, but not limited to, acne, hirsutism, alopecia, androgenic alopecia, inflammatory scalp alopecia, psoriasis, discoid lupus erythematosus, inflamed cysts, dermatologic manifestations of polycystic ovary syndrome, atopic dermatitis, pyoderma gangrenosum, pemphigus vulgaris, bullous pemphigoid, systemic lupus erythematosus, dermatomyositis, inflammatory vasculitis, sarcoidosis, Sweet's disease, type 1 reactive leprosy, capillary hemangiomas, contact dermatitis, lichen planus, exfoliative dermatitis, erythema nodosum, toxic epidermal necrolysis, erythema multiform, cutaneous T-cell lymphoma, seborrheic conditions, such as seborrheic dermatitis, oily skin or other conditions where the production of sebum and/or the overproduction of keratinocytes may be implicated, and any combination thereof.

In certain embodiments of the present invention, acne may be treated by a method of the present invention. Decrease of acne may be detected by a visual reduction in the amount or severity of the acne and/or by a decrease in pain or discomfort associated with the acne, as identified by the subject. In some cases, decreasing sebum production in the skin, particularly in those subjects that have an overproduction of sebum, may decrease, eliminate, and/or prevent acne.

In certain embodiments of the present invention, hair loss in a subject may be treated by a method of the present invention. Hair loss, also referred to as alopecia, that may be treated according to a method of the present invention includes, but is not limited to, male pattern hair loss, female pattern hair loss, inflammatory scalp alopecia, and any combination thereof. In some embodiments, a method of the present invention may increase hair follicles and/or slow the hair cycle. In certain embodiments, an androgen, such as, but not limited to, dihydrotestosterone (DHT) and/or testosterone, and/or an androgen related enzyme, such as Type II 5 α reductase, may be locally modulated by a method of the present invention.

In some embodiments, the delivery of nitric oxide is provided through administration of a nitric oxide releasing pharmaceutical composition of the present invention to the skin of a subject, including, but not limited to, a mucous membrane (including a body cavity), nail, and/or scalp of the subject. Any portion of a subject's skin may be treated. However, in some embodiments, a subject's face is treated by a method of the present invention. Furthermore, in some embodiments, a subject's trunk is treated by a method of the present invention. In some embodiments, a subject's scalp is treated by a method of the present invention. In certain embodiments, nitric oxide is delivered to the skin of a subject through topical administration of a nitric oxide releasing pharmaceutical composition of the present invention.

In some embodiments of the invention, a pharmaceutically acceptable composition may be administered to the skin via spray delivery. A non-aqueous delivery propellant may be used for water sensitive NO-releasing compounds such as diazeniumdiolate-modified compounds. Further, in some embodiments, particular components of the medicaments may be separated at some point prior to application of the medicament. For example, a water reactive NO-releasing compound may be stored separately from an aqueous component or propellant until application (e.g., via spraying or applying a gel). In some embodiments, the NO-releasing compounds may be combined with an aqueous constituent prior to application or the NO-releasing compounds and an aqueous constituent may be applied to the skin sequentially.

In some embodiments, an ointment containing nitrosothiol-modified compounds may be kept at a low temperature (e.g., <0° C.) to minimize thermal decomposition and NO release. The cold ointment may then be applied to the skin, and the elevated temperature of the skin may allow for the release of NO. In some embodiments, the nitrosothiol may be present in a medicament (e.g., a hydrophilic formulation which may limit NO diffusion) such that it is stable at room temperature due to cage effects, and then releases NO upon application to the skin. Light may also be applied to a medicament that includes nitrosothiol modified compounds. The application of light in fluxes may be applied to create fluxes of NO.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention will be further illustrated by the following non-limiting examples.

### EXAMPLES

A topical gel incorporating macromolecular delivery of nitric oxide was produced and tested as described below.

### Example 1

### Macromolecular Nitric Oxide Releasing Compound

A nitric oxide releasing macromolecular compound comprising MAPS was fabricated as described in United States Patent Application Publication No. 2009/0214618 and in PCT Patent Application Number PCT/US12/22048, filed Jan. 20, 2012, entitled “Temperature Controlled Sol-Gel Co-Condensation,” the disclosures of which are incorporated herein by reference as if set forth in their entirety. The resulting macromolecular particle were ball milled to provide an average particle size of from 8 to 10 μm to provide an active pharmaceutical ingredient (API).

### Example 2

### Topical Gel with Active Pharmaceutical Ingredient

Topical gel incorporating the API of Example 1 (“Active”) was fabricated as described in U.S. Provisional Patent Application Ser. No. 61/504,628 entitled “Topical Compositions and Methods of Using Same to Treat Acne,” and U.S. Provisional Patent Application Ser. No. 61/504,626, entitled “Methods of Manufacturing Topical Compositions and Apparatus for Same,” both of which were filed Jul. 5, 2011 and are incorporated herein as if set forth in their entirety. In particular, the Active had the formulation of:

TABLE 1
Active Gel

| | | Quality | | |
|---|---|---|---|---|
| Component | Supplier | Standard | Function | % w/w |
| Ethyl alcohol, anhydrous, 200 proof, | Koptec | USP | Solvent | 83.5 |
| USP | or | | | |
| | BDH | | | |
| Hexylene glycol, NF | Fluka/ | Reagent | Co-solvent/ | 10.0 |
| | Sigma | | Humectant | |
| Cyclomethicone, NF | Dow | NF | Water-repelling | 2.5 |
| ST-Cyclomethicone-5 | Corning | | agent | |
| Hydroxypropyl cellulose, NF | Ashland | NF | Viscosity- | 2.0 |
| Klucel MF Pharm | | | modifying agent | |
| NO Releasing Co-Condensed Silica | Novan | N/A | API | 2.0 |
| (Nitricil ™ NVN1) | | | | |

| | |
|---|---|
| Total | 100.0 |

Table 2 shows the in vitro nitric oxide release data for the 2% Nitricil™ NVN1 formulation.

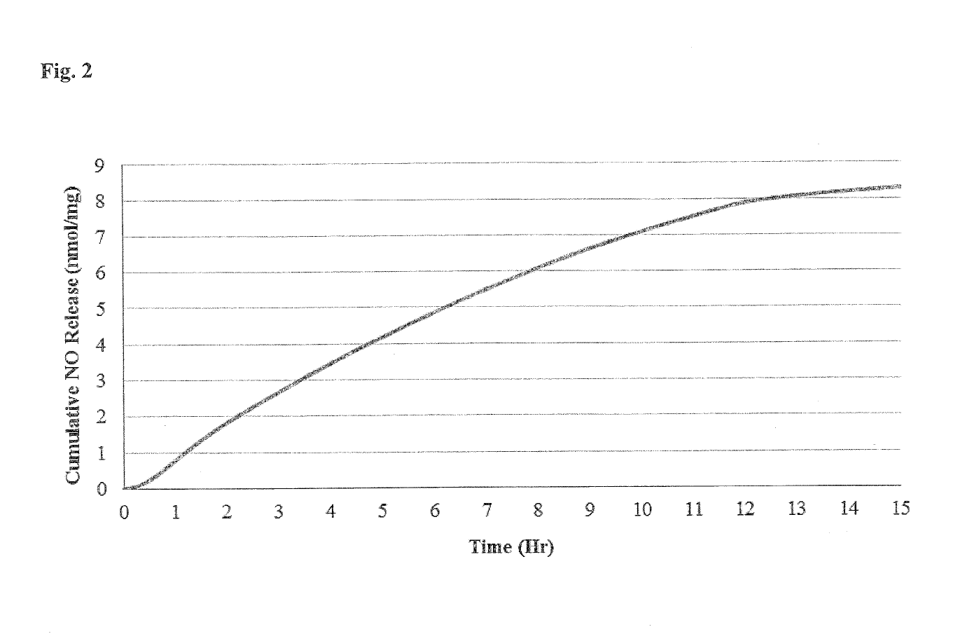

FIG. 2 shows the in vitro nitric oxide release profile for the 2% Nitricil™ NVN1 formulation over time.

Table 2 shows the in vitro nitric oxide release data for the 2% Nitricil™ NVN1 formulation.

| | | Cumulative | | | | |
|---|---|---|---|---|---|---|
| Sample | Cₘₐₓ | NO | Tₘₐₓ | T₅₀ | % | |
| Name | (nmol/mg · s) | (nmol/mg) | (min) | (min) | Total | pH |
| Nitricil ™ | 0.00034 | 9.1 | 66 | 330 | 9.1 | 10.5 |
| NVN1 | | | | | | |
| Gel, 2% | | | | | | |

### Example 3

### Topical Gel with Masking Agent

Topical gel incorporating titanium dioxide as a masking agent (“Vehicle”) was fabricated as described in U.S. Provisional Patent Application Ser. No. 61/504,628 entitled “Topical Compositions and Methods of Using Same to Treat Acne,” and U.S. Provisional Patent Application Ser. No. 61/504,626, entitled “Methods of Manufacturing Topical Compositions and Apparatus for Same,” both of which were filed Jul. 5, 2011 and are incorporated herein as if set forth in their entirety. In particular, the Vehicle had the formulation of:

TABLE 3
Vehicle Gel

| Component | Function | % w/w |
|---|---|---|
| Ethyl alcohol, anhydrous, | Solvent | 85.45 |
| 200 proof, USP | | |
| Hexylene glycol, NF | Co-solvent/Humectant | 10.0 |
| Cyclomethicone, NF | Water-repelling agent | 2.5 |
| ST-Cyclomethicone-5 | | |
| Hydroxypropyl cellulose, NF | Viscosity-modifying agent | 2.0 |
| Klucel MF Pharm | | |
| Titanium dioxide | | 0.05 |

| | |
|---|---|
| Total | 100.0 |

### Example 4

### Clinical Study Protocol

A single-center, double-blind, randomized, vehicle-controlled, parallel group study was conducted in 70 intent-to-treat (ITT) subjects with moderate to severe acne vulgaris. The study included male and female subjects between the ages of 12 and 40 (inclusive) with moderate to severe acne vulgaris, defined as at least 20 but no more than 40 inflammatory lesions (papules and pustules), 20 to 60 non-inflammatory lesions (open and closed comedones), no more than 2 nodules, and an Investigator Global severity Assessment of 3 or 4. Subjects who satisfied the entry criteria at the Baseline visit were randomized to either Active or Vehicle in a 1:1 ratio. Tolerability and safety assessments included cutaneous tolerability evaluation, physical exams, collection of vital signs, blood pressure monitoring, urine pregnancy tests (UPTs), blood methemoglobin monitoring and adverse event collection. Efficacy assessments included inflammatory and noninflammatory lesion counts and an Investigator global severity assessment

(IGA). Subjects returned for post-baseline evaluation at Weeks 2, 4, 6 and 8. Of the 70 ITT subjects enrolled in the study, 60 subjects completed the study per the study protocol.

### Example 5

### Tolerability and Safety Results

Cutaneous tolerability results of the study are shown in Table 4.

TABLE 4
Tolerability Results for ITT Active Subjects

| | Maximum | | | Week 8 Treatment | | |
|---|---|---|---|---|---|---|
| | Severity | | | Severity | | |
| | (N = 35) | | | (N = 35) | | |
| Local Reaction | Mild | Mod | Severe | Mild | Mod | Severe |
| Erythema | 12.1% | 5.7% | 0 | 3.0% | 0 | 0 |
| Scaling | 5.7% | 5.7% | 0 | 0 | 3.0% | 0 |
| Dryness | 0 | 0 | 0 | 0 | 0 | 0 |
| Itching | 11.8% | 6.1% | 0 | 6.1% | 6.1% | 0 |
| Burning/Stinging | 3.0% | 3.0% | 0 | 3.0% | 0 | 0 |

Methemoglobin results were obtained by pulse co-oximeter from Masimo. The results at baseline, 2 weeks and 8 weeks are shown in Table 5.

TABLE 5
Methemoglobin Levels as Measured by Pulse Co-Oximetry

| | Baseline | 2 Weeks | 8 Weeks |
|---|---|---|---|
| Active | | | |
| N | 35 | 35 | 34 |
| Mean | 0.9 | 1.1 | 0.9 |
| STD | 0.3 | 0.3 | 0.3 |
| Vehicle | | | |
| N | 35 | 34 | 31 |
| Mean | 0.7 | 1.0 | 0.8 |
| STD | 0.3 | 0.3 | 0.4 |

As seen in Table 5, the methemoglobin levels for Active and Vehicle subjects were will within acceptable ranges and did not differ significantly.

### Example 6

### Lesion Count and IGA Results

Inflammatory, non-inflammatory and total lesion counts were evaluated at baseline, 2 weeks, 4 weeks and 6 weeks. The average lesion counts for inflammatory lesions and non-inflammatory lesions are shown in Table 6.

TABLE 6
Lesion Counts for Per Protocol Subjects

| | | | 4 | 6 | 8 |
|---|---|---|---|---|---|
| Lesion Type | Baseline | 2 Weeks | Weeks | Weeks | Weeks |
| Inflammatory | | | | | |
| Active (N = 33) | 26.7 | 18.0 | 13.5 | 11.1 | 10.4 |
| Vehicle (N = 27) | 25.8 | 15.9 | 12.2 | 10.3 | 9.1 |
| Non-inflammatory | | | | | |
| Active (N = 33) | 34.9 | 28.2 | 21.1 | 18.1 | 18.2 |
| Vehicle (N = 27) | 35.4 | 27.7 | 23.1 | 20.3 | 19.3 |

### Example 7

### Non-Inflammatory Lesion Count Results by Gender

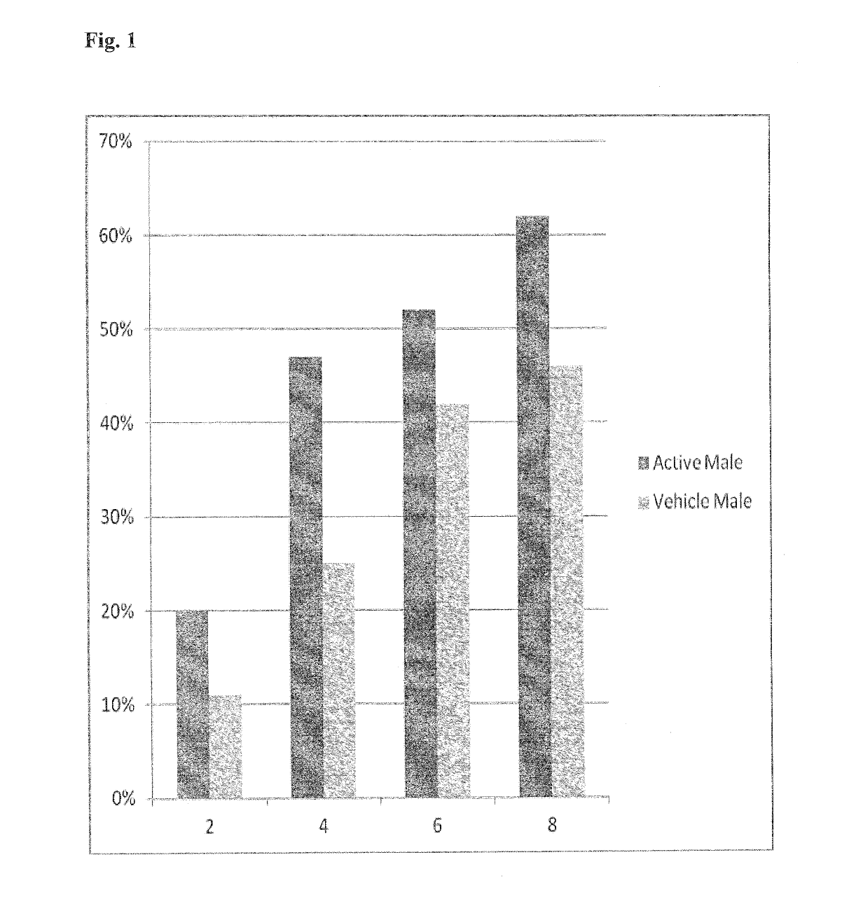

Of particular interest was the impact on non-inflammatory lesions in the male population in the study. Without wishing to be bound to any particular theory, in light of the connection between androgen activity, sebocytes and keratinocytes, the male populations, which may having increased androgen levels, may more readily exhibit changes in the disease state as a result of a reduction in the androgen activity at a pilosebaceous follicle. For example, males typically overproduce sebum at a greater rate than females, possibly as a result of, among other things, over expression of androgen local to the skin Thus, without being limited to a particular theory, a disproportionate reduction in non-inflammatory lesions associated with overproduction of sebum in the male population could indicate that the Active was affecting androgen activity in the skin of the male subjects, for example, by reducing androgen synthesis in the skin or inhibiting androgen affects on sebocytes or keratinocytes. The results of the clinical study for non-inflammatory lesion reduction are shown in Table 7 and a comparison of the male Active and Vehicle results is shown in FIG. 1.

TABLE 7
Non-inflammatory Lesion Reduction of Per Protocol Subjects by Gender

| Gender | Treatment | 2 Weeks | 4 Weeks | 6 Weeks | 8 Weeks |
|---|---|---|---|---|---|
| Male | | | | | |
| N = 9 | Active | 20% | 47% | 52% | 62% |
| N = 8 | Vehicle | 11% | 25% | 42% | 46% |
| Female | | | | | |
| N = 24 | Active | 21% | 39% | 47% | 44% |
| N = 19 | Vehicle | 25% | 36% | 38% | 43% |

As seen in Table 7, despite the small number of male subjects in the study, a pronounced difference in the reduction of non-inflammatory lesions between Active and Vehicle is seen at all time points in the male population. In contrast, in the female population a substantial difference between Active and Vehicle is seen only at the 6 week time point. Without wishing to be limited to a particular theory, this gender difference may suggest that the effect on androgen activity of the nitric oxide released from the API is more visible in the subjects where androgens play a greater role in the disease pathology, namely the male populations.

### Example 8

### Evaluation in the Hamster Flank Gland Model

A Nitricil™ gel and hydrogel were evaluated in the hamster flank gland model. The gel contained the active agent, Nitricil™ NVN1, and the hydrogel was used to aid in the release of nitric oxide from the gel. The Nitricil™ gel and hydrogel were stored refrigerated (2-8° C.) and prior to topical dosing were allowed to warm to room temperature. Each week a new tube of the gel or hydrogel was opened and used for the 7-days of dosing for that week. A water:ethanol (1:4) solution was used as the control (Vehicle).

Golden Syrian hamsters (Crl:LVG(SYR)) were used in the study. Golden Syrian hamsters have paired pelvic flank glands that respond to androgens by growing in size during puberty in response to increasing androgen synthesis and by maintaining gland size in adulthood, whereas antiandrogens limit growth and/or maintenance of pelvic flank gland size. The androgen-sensitive components of the flank organ include dermal melanocytes, sebaceous glands, and hair follicles.

A total of 30 male, 4-5 week old hamsters were assigned to the study. The animals were obtained from Charles River Laboratories or another acceptable source. The animals were laboratory bred and experimentally naive at the outset of the study. The hamsters weighed approximately 80-100 grams at the time of experimental start.

Hamsters were housed individually in plastic boxes containing corn cob bedding and equipped with water bottles. Primary enclosures were as described in the “Guide for the Care and Use of Laboratory Animals” (National Academy Press, Washington, D.C., 2010). The room was well ventilated (>10 air changes per hour) with 100% fresh air (no air recirculation). A 16-hour light/8-hour dark photoperiod was maintained to ensure maximum stimulation of sexual characteristics. Room temperature and relative humidity were maintained between 68-78° F. and 30-70%, respectively. Animal room cleaning was performed according to test facility SOPs.

Harlan Teklab Global Diet® 2016 was available ad libitum. No contaminants were known to be present in this certified diet that would interfere with the results of the study. Analysis of chow was limited to that performed by the manufacturer. Tap water was available ad libitum via water bottles. The water was routinely analyzed for contaminants.

Prior to assignment to the study, animals were selected for the study based on a health screen in accordance with good veterinary practice. Animals judged acceptable during the prestudy health screen were assigned to treatment groups. Animals were acclimated to laboratory conditions for approximately four days prior to the start of dosing.

Treatment of the animals was in accordance with regulations outlined in the USDA Animal Welfare Act (9 CFR Parts 1, 2 and 3) and/or the conditions specified in the “Guide for Care and Use of Laboratory Animals” (National Academy Press, Washington, D.C., 2011). A veterinarian, or designated technician, observed each animal at least twice daily for signs of illness or distress and reported any problems to the Study Director.

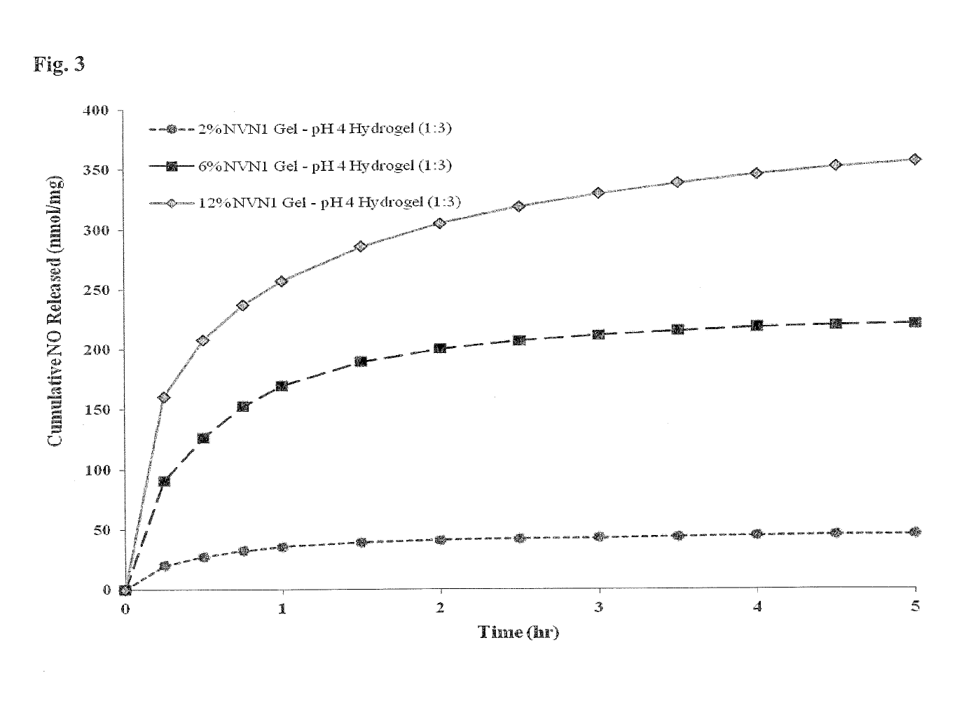

Table 8 shows the treatment groups included in the study. Table 9 shows the composition of the Nitricil™ NVN1 gel formulations, and Table 10 shows the composition of the hydrogel (pH 4). Table 11 shows the in vitro nitric oxide release data for each formulation upon mixing with the hydrogel at pH 4. FIG. 3 shows the in vitro nitric oxide release profile for the 2%, 6%, and 12% Nitricil™ NVN1 gel formulations upon mixing with the hydrogel at pH 4 over time. For the in vitro release data, 50 mg of the Nitricil™ NVN1 Gel sample and approximately 150 mg of the hydrogel (pH 4), for a ratio of the gel sample to hydrogel of 1:3, were transferred to a single, pre-cut weigh boat without allowing contact between the samples. The two samples were mixed for approximately 5-10 seconds, and then immediately placed into a clean, dry 50 mL NO measurement cell maintained at 37° C. with a moist nitrogen flow rate of 112-115 ml/min. The real-time in vitro release of nitric oxide for each mixture of the Nitricil™ NVN1 Gel sample and hydrogel was determined using chemiluminescence. Conversion from PPB NO to moles NO was achieved by measuring the NO generated from a known amount of sodium nitrite in a solution of potassium iodide to acquire a PPB-to-mole conversion factor. Any gaps in real-time NO release data resulting from multichannel operation were filled in by using a linear interpolation program. For any sample that was not measured to exhaustion of NO, a linear extrapolation to zero release of the last ˜5000 sec of release was performed. Real-time NO release data was then integrated, resulting in a total NO accumulation curve. NO release parameters such as Cₘₐₓ, Tₘₐₓ, Cumulative NO Released, and Time to Half of Total Released (T₅₀) were calculated from both the real time and total accumulation NO release curves. All calculations were performed automatically in custom-built data processing software (NovanWare v 1.05).

TABLE 8
Treatment groups included in the hamster flank gland model study.

| | | | Conc. | Dose | | No. |
|---|---|---|---|---|---|---|
| Grp | Treatment | Route | mg/mL | μL/animal | Schedule | hamsters |
| 1 | Vehicle | Topical | NA | 20/60 | NA | 6 |
| 2 | Gel Vehicle + | Topical | NA | 20/60 | Once daily × | 6 |
| | Hydrogel (pH 4) | | | | 4 wks | |
| 3 | Nitrici ™ NVN1 | Topical | 2% | 20/60 | Once daily × | 6 |
| | Gel Low Dose + | | | | 4 wks | |
| | Hydrogel (pH 4) | | | | | |
| 4 | Nitricir ™ NVN1 | Topical | 6% | 20/60 | Once daily × | 6 |
| | Gel Mid Dose + | | | | 4 wks | |
| | Hydrogel (pH 4) | | | | | |
| 5 | Nitricil ™ NVN1 | Topical | 12% | 20/60 | Once daily × | 6 |
| | Gel High Dose + | | | | 4 wks | |
| | Hydrogel (pH 4) | | | | | |

TABLE 9
Composition of the 2%, 6%, and 12% Nitricil ™ NVN1 gels.

% w/w

| | Gel | | | |
|---|---|---|---|---|
| Component | Vehicle | 2% | 6% | 12% |
| Isopropyl alcohol | 85.5 | 83.5 | 80.5 | 74.5 |
| Hexylene glycol | 10.0 | 10.0 | 10.0 | 10.0 |
| Cyclomethicone | 2.5 | 2.5 | 2.5 | 2.5 |
| Hydroxypropyl cellulose | 2.0 | 2.0 | 1.0 | 1.0 |
| Nitricil ™ NVN1 | 0 | 2.0 | 6.0 | 12.0 |

TABLE 10
Composition of the hydrogel with a pH of 4.

| Component | % w/w |
|---|---|
| Purified water | 89.1 |
| Glycerin | 10.0 |
| Carbopol ® 974P | 0.5 |
| Sorbic acid | 0.2 |
| Trolamine | 0.2 |

TABLE 11
In vitro nitric oxide release data for the 2%, 6%, and 12% Nitricil ™ NVN1 gels
upon mixing with the hydrogel at pH 4.

| | | Cumulative | | | | |
|---|---|---|---|---|---|---|
| | Cₘₐₓ | NO | Tₘₐₓ | T₅₀ | % | pH of |
| Sample Name | (nmol/mg · s) | (nmol/mg) | (min) | (min) | Total | Mixture |
| 2% Nitricil ™ NVN1 | 0.118 | 86 | 1 | 12 | 86 | 6.6 |
| Gel/Hydrogel (1:3) | | | | | | |
| 6% Nitricil ™ NVN1 | 0.240 | 220 | 1 | 22 | 73 | 6.6 |
| Gel/Hydrogel (1:3) | | | | | | |
| 12% Nitricil ™ NVN1 | 0.357 | 364 | 5 | 28 | 61 | 8.4 |
| Gel/Hydrogel (1:3) | | | | | | |

Treatments were applied topically once daily for 28 days to the right flank gland, leaving the left flank gland untreated. Prior to the first treatment, hair on the lower back of each animal was shaved to expose the flank glands. During the study shaving was conducted at intervals needed to ensure exposure of the flank glands to the test article and vehicle. The water/ethanol vehicle (20 μL) was applied to the right flank gland of the animals in Group 1. The test gel or vehicle gel (20 μL) at the appropriate concentration was applied to the right flank gland of each animal in Groups 2-5. Hydrogel (60 μL) was immediately applied on top of the test gel or vehicle gel and spread evenly over the dose site. All applications were made using a Pipetteman equipped with a polypropylene disposable tip. Prior to each subsequent application, the flank gland of each animal was wiped with an alcohol pad to remove residual compound. The left flank gland on each animal was not treated but was wiped with an alcohol pad each day to simulate removal of residual compound.

Gland areas were measured on Days 0, 14, 21 and 28. The sizes of the right and left flank glands on each animal were determined by measuring the length and width of the pigmented spot with a digital caliper (Fisherbrand digital calipers, Model 14-648-17). The surface area (mm²) of each spot was calculated as the product of the two measurements. Gland measurements were recorded prior to dosing, once weekly during the study and just prior to terminal sacrifice.

The body weight of each animal was recorded prior to the start of the study and then twice weekly. All animals were observed once each morning and afternoon throughout the study for viability. A brief physical examination was performed on each animal at 1 hr postdose (i.e., the approximate Tmax for the Nitricil™ Gel) to document health status.

Blood was collected from each animal at the end of the study. Samples (target of 1 mL) were obtained via the orbital plexus into a tube without anticoagulant. The samples were kept on ice and transferred to the clinical pathology laboratory within 1 hr of collection for processing. The samples were allowed to clot and then centrifuged to obtain serum. The samples were kept on ice and centrifuged (within 1 hr) in a unit set at 5° C. for 10 minutes at 2000 xg. The cellular components and serum were separated and the cellular components were discarded. The serum was harvested into a single tube for each animal. The samples were stored in an ultra-low freezer set at −70° C. pending transfer to the analytical lab for possible future analysis.

After blood collection the animals were euthanized by asphyxiation with CO₂ and photographs were taken of the flank glands of representative animals from each group. The right and left flank glands and surrounding skin were then excised. A standard sample of gland and small amount of surrounding skin was cut from the excised skin using a biopsy punch. Each gland sample, left and right, was placed in a separate, labeled container of 10% formaldehyde for possible future analysis.

One-way ANOVA and Dunnett's post test was performed using GraphPad InStat (version 3.10, GraphPad Software, San Diego Calif. USA) to determine differences between the respective vehicle control and the treated animals. Significance was set at P<0.05 level.

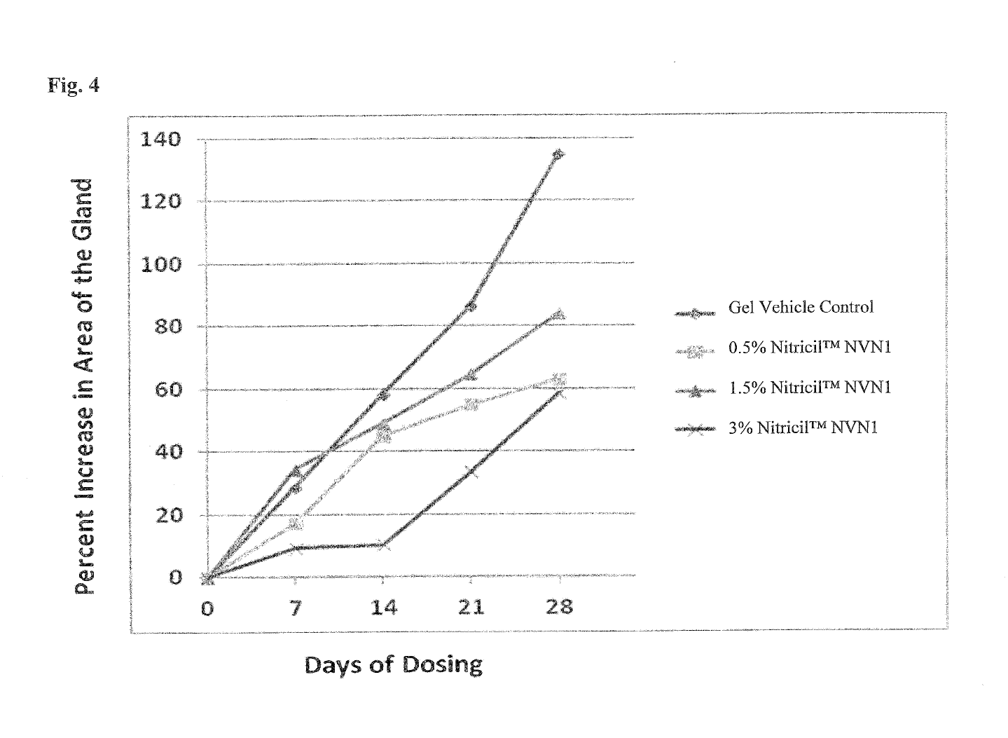

The mean flank gland areas of the vehicle and gel control groups were not significantly different at any of the measurement time points. Significant differences were first detected among the groups on Day 14 (Table 12). The mean gland area of the 12% Nitricil™ NVN1 group was significantly smaller than that of the gel control group at Day 14 (Table 12). On Day 21, the mean areas of the 6% and 12% Nitricil™ NVN1 groups were significantly smaller than the gel control group (Table 12). At the final measurement on Day 28, the mean gland areas for all three dose levels of Nitricil™ NVN1 were significantly smaller than the gel control group (Table 12). There were no significant differences among the groups in the size of the untreated left flank glands at any of the measurement time points (Table 12). FIG. 4 shows the percent change in treated gland area over the course of treatment. FIG. 5 shows the percent change in the right flank gland area after once daily treatment for 28 days.

TABLE 12
Mean flank gland areas (±SEM) in male Golden Syrian hamsters after treatment once daily for 28 days.

| Right flank | Day of study | | | | | | | | | |
|---|---|---|---|---|---|---|---|---|---|---|
| gland | 0 | | 7 | | 14 | | 21 | | 28 | |
| treatment | Left | Right | Left | Right | Left | Right | Left | Right | Left | Right |
| Solvent vehicle control | 39.3 ± 4.7 | 39.7 ± 4.7 | 40.1 ± 1.7 | 43.5 ± 2.9 | 45.7 ± 3.1 | 48.9 ± 4.2 | 54.0 ± 4.3 | 59.0 ± 3.7 | 67.5 ± 3.8 | 72.6 ± 3.1 |
| Gel vehicle control | 34.9 ± 3.4 | 32.2 ± 3.4 | 40.7 ± 2.5 | 41.5 ± 2.4 | 50.5 ± 3.1 | 51.0 ± 2.5 | 57.2 ± 1.5 | 60.1 ± 2.4 | 72.5 ± 1.4 | 75.7 ± 2.6 |
| 2% NVN1000 | 36.5 ± 2.7 | 35.4 ± 2.7 | 40.0 ± 3.1 | 41.4 ± 4.1 | 48.6 ± 4.4 | 51.2 ± 3.1 | 57.1 ± 2.4 | 54.7 ± 2.3 | 66.4 ± 4.3 | 57.6 ± 3.2** |
| 6% NVN1000 | 34.2 ± 2.0 | 28.9 ± 2.0 | 40.4 ± 1.7 | 38.8 ± 1.9 | 43.0 ± 3.3 | 43.1 ± 4.2 | 54.3 ± 1.8 | 47.5 ± 2.1* | 65.9 ± 2.6 | 54.2 ± 2.4** |
| 12% NVN1000 | 35.1 ± 2.2 | 33.8 ± 2.2 | 39.1 ± 1.7 | 36.9 ± 1.8 | 44.2 ± 2.6 | 37.2 ± 2.8 | 58.9 ± 1.8 | 45.0 ± 3.0** | 68.3 ± 3.0 | 53.5 ± 3.1** |

*Indicates a significant difference from the gel vehicle control at P < 0.05,
**Indicates a significant difference from the gel vehicle control at P < 0.01.

All hamsters appeared normal throughout the study. No adverse signs were recorded at any of the one-hour post-dose observations. The hamsters steadily gained weight throughout the study. No significant differences in bodyweight were observed among the groups at any of the time points.

Nitricil™ NVN1 significantly inhibited the growth of the treated flank glands in the hamsters. Inhibition was first evident at Day 14 with the highest (12%) dose group of Nitricil™ NVN1 Gel. After 28 days of dosing, the treated flank glands for all three formulations of Nitricil™ NVN1 Gel were significantly smaller compared to the vehicle and solvent control groups. All hamsters gained weight over the course of the study and no adverse signs were observed.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method of treating a steroid hormone related disorder in the skin of a subject, the method comprising:
administering a nitric oxide-releasing pharmaceutical composition to the skin of the subject to locally modulate steroid hormone activity in the skin of the subject, thereby treating said steroid hormone related disorder in the skin of the subject.

2. The method of claim 1, wherein the nitric oxide-releasing pharmaceutical composition comprises at least one nitric oxide releasing active pharmaceutical ingredient.

3. (canceled)

4. The method of claim 2, wherein the at least one nitric oxide releasing active pharmaceutical ingredient comprises a diazeniumdiolate functional group.

5. The method of claim 2, wherein the at least one nitric oxide releasing active pharmaceutical ingredient comprises NO-releasing co-condensed silica particles.

6.-7. (canceled)

8. The method of claim 1, wherein the steroid hormone activity is locally modulated at a follicular steroid hormone target in the skin of the subject.

9. The method of claim 1, wherein locally modulating comprises decreasing steroid hormone activity.

10.-18. (canceled)

19. The method of claim 1, wherein the nitric oxide-releasing pharmaceutical composition is administered to the skin of the subject with no systemic nitric oxide effects.

20. A method of reducing steroid hormone induced sebum production in the skin of a subject, the method comprising:
administering a nitric oxide-releasing pharmaceutical composition to the skin of the subject to locally modulate steroid hormone activity in the skin of the subject, thereby reducing sebum production in the skin of the subject.

21. The method of claim 20, wherein the nitric oxide-releasing pharmaceutical composition comprises at least one nitric oxide releasing active pharmaceutical ingredient comprising a diazeniumdiolate functional group.

22.-23. (canceled)

24. The method of claim 21, wherein the at least one nitric oxide releasing active pharmaceutical ingredient comprises NO-releasing co-condensed silica particles.

25.-26. (canceled)

27. The method of claim 20, wherein the steroid hormone activity is locally modulated at a follicular steroid hormone target in the skin of the subject.

28. The method of claim 20, wherein locally modulating comprises decreasing steroid hormone activity.

29.-33. (canceled)

34. The method of claim 20, wherein the nitric oxide-releasing pharmaceutical composition is administered to the skin of the subject with no systemic nitric oxide effects.

35. A method of reducing proliferation and/or differentiation of sebocytes and/or keratinocytes in the skin of a subject, the method comprising:
administering a nitric oxide-releasing pharmaceutical composition to the skin of the subject to locally modulate steroid hormone activity in the skin of the subject, thereby reducing proliferation and/or differentiation of sebocytes and/or keratinocytes in the skin of the subject.

36. The method of claim 35, wherein the nitric oxide-releasing pharmaceutical composition comprises at least one nitric oxide releasing active pharmaceutical ingredient.

37. (canceled)

38. The method of claim 36, wherein the at least one nitric oxide releasing active pharmaceutical ingredient comprises a diazeniumdiolate functional group.

39. The method of claim 36, wherein the at least one nitric oxide releasing active pharmaceutical ingredient comprises NO-releasing co-condensed silica particles.

40.-41. (canceled)

42. The method of claim 35, wherein the steroid hormone activity is locally modulated at a follicular steroid hormone target in the skin of the subject.

43. The method of claim 35, wherein locally modulating comprises decreasing steroid hormone activity.

44.-49. (canceled)

50. The method of claim 35, wherein the nitric oxide-releasing pharmaceutical composition is administered to the skin of the subject with no systemic nitric oxide effects.
